(19) United States
(12) Patent Application Publication
(10) Pub. No.: US 2022/0288094 A1
(43) Pub. Date: Sep. 15, 2022

(54) COMPOSITIONS AND METHODS FOR IMMUNE MODULATION OF PATHOLOGIC INFLAMMATION

(71) Applicants: Tanya Kuritz, Kingston, TN (US); John Patrick Biggerstaff, Banbury (GB)

(72) Inventors: Tanya Kuritz, Kingston, TN (US); John Patrick Biggerstaff, Banbury (GB)

(21) Appl. No.: 17/343,047

(22) Filed: Jun. 9, 2021

(51) Int. Cl. A61K 31/66; A61P 37/00

(52) U.S. Cl. CPC A61K 31/66; A61P 37/00

(57) ABSTRACT

The invention describes modulating the innate immune response to pathogens by the compounds comprising C35-C90 polyisoprenyls of Formula 1 and Formula 2, as well as their derivatives, as well as methods of their use, including activation and or modulation of the integrins, including those with CD11b chain. Such modulation consists of the upregulation of integrins and attenuation of pathological inflammation in a disease while sustaining the innate antiviral response via Toll-receptors. This invention is directed towards using the above as antimicrobial, immunoregulatory, anti-cancer, anti-inflammatory treatments for humans and animals, including in diseases where pathological inflammation is caused by coronaviruses (SARS-CoV-2, FIPV), herpesviruses, and other agents.

### FIELD OF THE INVENTION

The invention relates to the applications of compositions for the modulation of the integrin-mediated immune response and modulation of pathologic inflammation.

### BACKGROUND OF THE INVENTION

Despite numerous drug development efforts in oncology and infectious diseases, chemotherapy, including direct antiviral drugs, showed limited utility for treating those diseases because it does not address pathological inflammation, which is a side effect of the pathogen-triggered immune response. The redirection of the host immune response into a pathological inflammation is an adaptational feature of pathogens. Most viral and oncological pathologies in humans and animals are due to the pathological inflammation of vital organs caused by those agents. The drug discovery is refocusing on finding methods and compositions modulating pathological immune responses. The recent epidemics of COVID-19 underscored the critical necessity of being armed with a selection of different drugs, especially those modulating the innate immune response to the virus and attenuating pathological inflammation.

The immune system defends the organism against pathogens. The defense response aims to protect the organism's function by neutralizing or eliminating pathogens. It consists of a complex cascade of events triggered by pathogen exposure and involves both pathogen-specific and non-specific mechanisms. The non-specific, innate immune response is the first line of defense. However, pathogens evolved ways to counter the innate response by evading and weakening it.

Viruses and oncogenes disrupt the innate immune response, and in most cases, this disruption involves the overproduction (termed as burst or storm) of proinflammatory cytokines, which changes normal cellular functions and restructures pathways causing pathological inflammation and the associated disease (Germolec, et al., Methods Mol. Biol., 2018, 1803: 57-79). Antiviral drugs target the virus entry or replication and do not address the inflammation in the host, and the latter may persist in the absence of the viral load. Most anti-inflammatory drugs and chemotherapy methods reduce inflammation by suppressing all immune response pathways, including beneficial innate immunity protections. The approach causes an increase in the viremia and often worsens the pathology due to the concurrent inhibition of the antiviral innate cellular response (Rhen, Cidlowski, NEJM, 2005, 335, 1711-1723).

The herpesviruses and coronaviruses are ubiquitous pathogens that cause diseases in humans and animals. Examples include human coronaviruses, e.g., SARS-CoV-2, the virus causing COVID-19, and feline enteric coronavirus (FCoV) that causes feline infectious peritonitis (FIP), a fatal disease. Another example is human herpes viruses causing different clinical disorders in humans and feline herpes viruses that cause rhinotracheitis. Those and other retroviruses are recognized by innate immune receptors and induce inflammatory central to the pathogenesis.

The dysregulation of the host immunity leading to the cytokine burst and subsequently pathological inflammation was described, for example, for herpesvirus-triggered diseases in humans (Ma, He, J. Mol. Biol., 2014, 426: 1133-1147) and in cats (Nelli et al., Virus Res., 2016, 214: 39-48). In another example, coronavirus-induced proinflammatory cytokine storms lead to inflammation and disease, such as COVID in humans (Mehta et al., Lancet, 2020, 395: 1033-1034) and feline infectious peritonitis in cats (Malbon et al., J. Comp. Path, 2019, 166: 69-86, Pedersen, Vet. J., 2014, 201: 23-132).

Hence, the innate immune response to pathogens, including the viruses, consists of at least two separate components:

(1) beneficial response: upregulation of the proinflammatory cytokines to levels that enable host defense, and

(2) pathological (disruptive) response: triggering an overproduction of the proinflammatory cytokines by the pathogen, which results in clinical pathological inflammation and disease.

Therefore, the desirable method for disease control and treatment should be a correction of the aberrant immune response; it should reduce the excessive inflammation while supporting antiviral innate immune response by the same cytokines (Wolf et al., Nature Comms., 2018, 9: 525, doi:10.1038/s41467-018-02896-8). Reed (2014, in: Canine and Feline Respiratory Medicine, L. R. Johnson, Ed. Elsevier, Philadelphia, Pa.) underscores the need for “more targeted therapies to modify this inflammatory response.”

Inflammation damages organs and tissues and affects different physiological processes. However, no single, specific test exists to measure the extent of the inflammation universally. The damages to the organism can be evaluated by standard clinical hematology and clinical chemistry tests in the blood; however, no clinical markers are exclusive or specific to the inflammation alone. For instance, anemia may be caused by inflammation, or blood loss, or metabolic disease, and the detection of anemia per se, in a clinical setting, cannot be used to diagnose inflammation (Fraenkel, Med. Clin. North. Am., 2017, 101: 285-296). In practice, a combination of different markers and clinical symptoms is required to diagnose the inflammation. Those markers include but are not limited to abnormal composition and concentration of various leukocytes, including neutrophilia, blood protein changes, and others (Germolec et al., Methods Mol. Biol., 2018, 1803: 57-79).

For instance, the coronavirus SARS-CoV-2-induced Th1-cytokine storm causes inflammation in COVID patients (Mehta et al., 2020, Lancet 395: 1033-1034). The most common findings characteristic of the cytokine storm and inflammation in the patients is neutrophilia (Frater et al., Int. J. Lab. Hematol., 2020, 42, Suppl. 1: 11-18). Neutrophilia is considered “an expression of the cytokine storm and hyperinflammatory state which have an important pathogenetic role in COVID-19 and related infections such as SARS” (ibid.) and is most commonly found in severe COVID cases (Chen et al., EMBO J., 2020, 39: e105896). Furthermore, feline infectious peritonitis (FIP), a fatal coronaviral disease of cats, is also associated with the cytokine storm and Th-1 pathways dysregulation (Watanabe et al., Vet, Res. 2018, 49: 81; Malbon et al., 2019, J. Comp. Path, 166: 69-86). The probable diagnosis is based on clinical presentation and multiple changes in non-specific blood makers, including the decrease in albumin/globulin ratio lymphopenia, neutrophilia, leukocytosis, anemia, etc.; however, the definitive diagnosis requires histological evidence of pyogranulomatous inflammation of the internal organs, commonly lymph nodes (Addie et al., 2009, J. Feline Med. Surg., 2009, 11: 594-604).

For example, human and feline diseases caused by coronaviruses, e.g., SARS and FIP, respectively, have similar clinical pathologies and immune pathologies that involve a virus-induced dysregulation of the immune response (deGroot Minjes et al., J. Virol., 2005, 79: 1046-1044). Furthermore, the pathological basis is similar in the diseases caused by herpes viruses in humans (HSV; Brun et al. Front. Microbiol. 2018, 9:2148 doi: 10.3389/fmicb.2018.02148) and cats (FHV; Lee et al., Virus Res., 2019, 264: 56-67).

Hence, along with clinical evidence, neutrophilia is an accepted but not an exclusive means for detecting coronavirus-triggered cytokine storms. At the same time, the presence of pyogranulomatous inflammation of the internal organs confirms the diagnosis in cats.

Recent research identified different signal transduction pathways involved in the immune response to pathogens. The cellular adhesion and signal transduction molecules called integrins are central for many of those pathways. Integrins are heterodimeric transmembrane proteins comprised of alpha and beta subunits; 24 combinations are currently known (Raab-Westphal et al., Cancers, 2017, 9, 110). Further research and development produced integrin-targeted pharmaceuticals, which inhibit integrins or integrin ligands involved in the disruptive response (Millard et al., Theranostics, 2011, 1: 154-188, Wolf et al., Nature Comms., 2018, 9: 525).

The known integrin-targeting substances are either monoclonal antibodies or peptides, which either inhibit integrins directly or by blocking their ligands (Raab-Westphal et al., Cancers, 2017, 9, 110). Several integrin-targeting inhibitors were approved or are currently in trials for infectious or oncological diseases (ibid.), including for SARS-2 virus, the cause of COVID-19 (Beddingfield el al., Basic to Translational Sci., 2021, 6: 1-8).

The integrin subunit alpha M (ITGAM), also known as cluster of differentiation (CD) molecule 11b, and complement receptor (CR)3A, is constitutively expressed on leukocytes of the innate immune system, e.g., monocytes, granulocytes, macrophages, and natural killer cells (Solovjev et al., J. Biol. Chem., 2005, 280: 1336-1345). CD11b integrin chain (subunit) forms various heterodimeric complexes with other integrins chains (subunits). These multifunctional heterodimers are involved in cross-talk with elements of innate and acquired immunity; they serve as receptors, adhesion molecules, and signal regulators in various immunity pathways (Bednarczyk et al., Int. J. Mol. Sci., 2020, 21, 1402).

Single nucleotide polymorphisms in the human CD11b gene correlated with the clinical severity of systemic lupus erythematosus, whereas CD11b activation suppressed it (Faridi et al., J. Clin. Invest., 2017, 127: 1271-1283). Using the murine knock-out mutants, Zerria et al. (Immunol., 2006, 119: 431-440) showed that the recombinant A-domain of CD11b blocked diapedesis and protected tissue from damage in an inflammatory model of a muscular injury. CD11b also modulated down the pathological inflammasome-mediated responses triggered by the human herpesvirus (Campadelli-Fulmi et al., 2018).

CD11b is activated and functions independently of the TLR pathway; it may modulate TLR signaling by enhancing or inhibiting TLR-mediated responses depending on the nature of the cells and tissues (Han et al., Nature Immunol., 2010, 11: 734-741; Ling et al., Nature Comm., 2014, 5:3039, DOI: 10.1038/ncomms4039).

Phosphorylated polyprenols are established as an agent of upregulation of Th-1 mediated immunity, as patented in Danilov et al. (U.S. Pat. No.6,525,035) and Kuritz (U.S. Pat. No. 9,872,867). These compositions upregulated biosynthesis of the corresponding cytokines dependent on the functional Toll-receptors TLR-2 and TLR-4 (U.S. Pat. No. 9,872,867 to Kuritz) and were considered TLR agonists, and their action was attributed to the upregulation of Th-1 proinflammatory cytokines (Legendre et al., Front. Vet. Sci., 2017, 4: 7).

Here, we prove that the compositions act as an effector of the integrin CD11b. Furthermore, we show that the composition decreases pathological inflammation, which is likely due to integrin activation.

The development of new compositions and methods for modulation of immune responses is useful for preventing and or treating diseases and illnesses due to cytokine-related inflammation and researching the function of the integrins and cellular communications in innate immunity.

### SUMMARY OF THE INVENTION

The present invention relates to the use of various polyisoprenyls for the regulation of the innate immune response via integrins comprising the integrin chain CD11b and any other integrin chain. The compositions of phosphorylated polyprenols are incorporated by references to U.S. Pat. No. 6,525,035 to Danilov et al. and U.S. Pat. No. 9,872,867 to Kuritz.

The present invention relates to the use of the said compositions to decrease the pathological inflammation caused by the disruptive inflammatory immune response. In particular, the invention relates to the compositions' ability to upregulate and or modulate the integrin CD11b, which is involved in the cross-talk and regulation of the cytokine levels, independently of and or including TLR pathways.

Before this invention, the compositions were known for their ability to upregulate proinflammatory Th-1 type cytokines in the TLR-dependent manner (U.S. Pat. No. 9,872,867 to Kuritz). By extension, they were considered, by skilled in the art, as contributors to the enhanced cytokine response (Legendre et al., 2017) and inflammation. Furthermore, the ability of these compounds and pharmaceutical compositions based on these compounds to affect the integrins' expression was unknown. Moreover, the ability of these compounds to affect integrins was not obvious because all known integrin-targeting molecules have a different chemical nature, e.g., proteins and peptides. Furthermore, all integrin-targeting therapies involve direct inhibition of the integrins or direct inhibition of their ligands. Further still, the ability of these compounds and pharmaceutical compositions based on these compounds to reduce disease-related inflammation was also unknown. Further still, since the activation and or mediation of CD11b functionality is independent of other forms of immune responses, its link to the upregulation of proinflammatory cytokines by the compounds was not obvious. The resolution of pathological inflammation is also an independent physiological outcome. Hence, the activation of the CD11b pathway by the compounds of the present invention and reduction of the inflammation is novel and non-obvious over that already known by those practiced in the art.

Consequently, the ability of the compounds of the present invention (phosphorylated polyprenols) to modulate the innate immune response via intergins and to attenuate the inflammation was unknown at the time of the invention. Additionally, since the TLR-mediated induction of Th-1 proinflammatory cytokines is considered a cause of the inflammation in the disease (Germolec et al., Methods Mol Biol 2018, 1803: 57-79), the attenuation of the inflammation by the same compositions was not obvious. Furthermore, the induction of integrins may exacerbate pathological inflammation (Behera al., J. Immunol., 2006, 177: 657-674) and upregulate TLR-triggered sepsis (Chen et al., Sci. Rep., 2016, 6: 28841), and exacerbate sepsis lethality (Lerman el al., Blood, 2014, 124: 3515-3523). Further still, the pathological inflammation in feline infectious peritonitis was associated with the induction of integrins by the virus (Olyslaegers et al., Vet. Microbiol., 2013, 166: 438-449). Further still, integrins and TLR receptors recognize different patterns and are activated by different ligands, for example, in a herpes virus infection (Gianni et al., Proc. Natl. Acad. Sci., 2012, 109: 19792-19797). Hence, the upregulation of the integrins and induction of TLR-dependent cytokine burst are independent. Furthermore, the induction of both TLR- and integrin-dependent pathways by the same compound is non-obvious.

In this regard, the present invention is related to compounds based on the phosphorylated polyprenol architecture and the use of those compounds for the regulation of innate immune system functions and for the practice of research. In particular, the compounds of the present invention are useful as modulators of the innate immune response via, for example, the activation of integrins.

For the purposes of this application, the term “innate immune response,” “innate immunity,” and similar phrases are defined as a portion of the immune system comprising the cells and mechanisms that defend the host from infection by other organisms in an antigen-non-specific, on-antibody-mediated manner. This means that the cells of the innate system, including but not limited to dendritic cells, monocytes, macrophages, mast cells, neutrophils, recognize and respond to pathogens in a non-specific way and, unlike the adaptive immune system (comprising the humoral and adaptive immune responses), it does not confer long-lasting or protective immunity to the host. Innate immune systems provide immediate defense against infection and are responsible for, e.g., the inflammatory response.

Furthermore, for this application's purposes, as applied to the innate immune response, the term “beneficial response” or “beneficial immune response” refers to the component, which includes upregulation of the proinflammatory cytokine response to levels that sustain host defense. Further still, the term “disruptive response” or “disruptive immune response” refers to the overproduction (burst or storm) of proinflammatory cytokines, which cause pathological inflammation and disease.

Polyprenols and phosphorylated polyprenols (polyprenyls), also known as polyisoprenols and polyisoprenol phosphates (polyprenyls), are incorporated by reference to U.S. Pat. No. 9,872,867 to Kuritz. In this regard, it is contemplated that the present invention also comprises compounds and derivatives based on the phosphorylated polyprenol architecture presented below.

In this regard, the present invention contemplates compositions comprising,

1) polyprenol monophosphates of Formula 1:

R1—CH2(R2—CH2)—C—CH—CH2—[CH2—C(CH3)—CH—CH2]n-P(—O)(O)2—R3(R4),

wherein R1 and or R2 and or R3 and or R4 are selected from a group consisting of, —H, —OH, —CH3, hydrocarbons, amino acids, amines, lipids, carbohydrates, or sugars and wherein n is an integer from 5-18 inclusive or a salt thereof, and or
2) polyprenol pyrophosphates of the Formula 2:

R1(R2)—C═CH—CH2—[CH2—C(CH3)═CH—CH2]m-O—P(═O)(OH)—O—P(═O)(O)2—R3(R4),

wherein R1 and or R2 and or R3 and or R4 are selected from a group consisting of, —H, —OH, —CH3, hydrocarbons, amino acids, amines, lipids, carbohydrates, or sugars and wherein m is an integer from 5-18 inclusive or a salt thereof. The invention also provides pharmaceutical compositions comprising the compounds of the invention as well as therapeutic methods for using the compositions.

In another embodiment, one or both of R1 and R2 are not methyl groups (—CH3).

It will be appreciated by those skilled in the art that polyisoprenes possess double bonds which may exist in cis-, or trans-configurations. It is to be understood that the present invention encompasses any stereoisomeric form of the species as well as mixtures thereof, which possess the useful properties described herein.

The present invention also comprises compounds based on the phosphorylated polyprenol architecture above and wherein the terminal phosphate group is a salt and or additionally comprises a moiety selected from a group consisting of carbohydrates, lipids or metals or salts or acids thereof.

Compounds based on the phosphorylated polyprenols of the present invention (Formula 1 and or Formula 2) with the modifications listed in the preceding two paragraphs above are herein known as “derivatives of Formula 1,” “derivatives of Formula 2,” “derivatives,” “compound(s) of the present invention,” the “Substance” or the like.

The synthesis of derivatives of the phosphorylated polyprenols of Formula 1 and Formula 2 are known in the art and incorporated herein by reference to U.S. Pat. No. 9,872,867 to Kuritz.

In one embodiment, the compounds of the present invention comprise a pharmaceutically acceptable carrier. In this regard, when comprising a pharmaceutically acceptable carrier, the compound(s) of the present invention comprises about 50 wt % (weight percent), 75 wt %, 90 wt %, 95 wt %, about 99 wt %, about 99.5 wt %, about 99.9 wt % or greater of one or more of the derivatives given above.

In another embodiment, the compound of the present invention comprises about 95 wt %, about 99 wt %, about 99.5 wt %, about 99.9 wt % or greater of one of the derivatives given above. In another embodiment, when comprising a pharmaceutically acceptable carrier, the compound of the present invention comprises up to about 99.9% of one or more of the compositions of the present invention.

In another embodiment, when comprising a pharmaceutically acceptable carrier, the compound(s) of the present invention comprises not less than 90 wt %, 95 wt %, 99 wt %, or 99.9 wt % of one or more of the compositions of the present invention.

Pharmaceutical compositions comprising the compound(s) of the present invention may be used, for example, to modulate or stimulate the innate immune system of an animal or other organism. In this regard, the pharmaceutical composition comprising of an effective immunomodulatory (of the innate immune system) amount of one or more compounds of the present invention is administered to an animal (preferably a mammal) or another organism (herein collectively referred to as the “subject” or subjects”) in need of such treatment.

In another embodiment, pharmaceutical compositions comprising the compound(s) of the present invention may be used, for example, to modulate or stimulate the upregulation or activation of one or more integrins of an animal or other organism. In this regard, the pharmaceutical composition comprising an effective integrin-modulating amount of one or more compound(s) of the present invention is administered to an animal (preferably a mammal) or another organism (herein collectively referred to as the “subject” or “subjects”) in need of such treatment.

The present invention also contemplates that the compounds and compositions of the invention are used, for example, to screen for small molecule inhibitors and activators of integrin-mediated signaling or activity. For example, the compounds and compositions of the present invention may be used as positive controls in an assay system designed to screen for such small molecules effective in modulating integrin-mediated signaling or activity.

Furthermore, it is contemplated that the compounds and compositions of the present invention are useful to identify other receptors that may interact with the compounds and compositions of the present invention. In this regard, the compounds of the present invention may be tagged with molecules for detection via biochemical or biological means. For example, fluorescent tags may be used for detection via fluorescent microscopy or flow cytometry. Electron dense tags may be used for detection via electron microscopy.

Still further, the compounds and compositions of the present invention may be administered to research animals to investigate innate immune system modulation and signaling modulation via integrins.

Still further, the compounds and compositions of the present invention may be administered to research animals to investigate the modulation of inflammation via integrins.

In regards to the teachings of this specification, in one embodiment, the present invention contemplates a method of modulating innate immune responses via integrins in a subject, said method comprising administering to or causing to be administered to the subject a pharmaceutical composition comprising at least one polyprenol phosphate selected from the general formulas:

R1—CH2(R2—CH2)—C═CH—CH2—[CH2—C(CH3)═CH—CH2]n-P(═O)(O)2—R3(R4)

R1(R2)—C═CH—CH2—[CH2—C(CH3)═CH—CH2]m-O—P(═O)(OH)—O—P(═O)(O)2—R3(R4)

wherein R1 and or R2 and or R3 and or R4 are selected from a group consisting of, —H, —OH, —CH3, hydrocarbons, amino acids, amines, lipids, carbohydrates or sugars and wherein m or n is an integer from 5-18 or a salt thereof.

Further in regards to the teachings of this specification, in one embodiment, the present invention contemplates a method of attenuating of inflammation via integrins in a subject, said method comprising administering to or causing to be administered to the subject a pharmaceutical composition comprising at least one polyprenol phosphate selected from the general formulas:

R1—CH2(R2—CH2)—C═CH—CH2—[CH2—C(CH3)═CH—CH2]n-P(═O)(O)2—R3(R4)

R1(R2)—C═CH—CH2—[CH2—C(CH3)═CH—CH2]m-O—P(═O)(OH)—O—P(═O)(O)2—R3(R4)

wherein R1 and or R2 and or R3 and or R4 are selected from a group consisting of, —H, —OH, —CH3, hydrocarbons, amino acids, amines, lipids, carbohydrates or sugars and wherein m or n is an integer from 5-18 or a salt thereof.

In another embodiment, the present invention contemplates that the pharmaceutical composition additionally used in a method of modulating the functionality of integrins in a subject comprises a pharmaceutically acceptable carrier.

In yet another embodiment, the present invention contemplates compound(s) of the present invention used in a method of modulating the functionality of integrins in a subject, wherein n is 8, 9, 10, 11, 12, or 13 in greater than 50% of the polyprenol monophosphates.

In still yet another embodiment, the present invention contemplates compound(s) of the present invention used in a method of modulating the functionality of integrins in a subject, wherein m is 8, 9, 10, 11, 12, or 13 in greater than 50% of the polyprenol pyrophosphates.

In still yet another embodiment, the present invention contemplates compound(s) of the present invention used in a method of modulating the functionality of integrins in a subject, wherein the weight percent of polyprenol monophosphates is greater than the weight percent of the polyprenol pyrophosphates.

In still yet another embodiment, the present invention contemplates compound(s) of the present invention used in a method of modulating the functionality of integrins in a subject, wherein the weight percent of polyprenol monophosphates is not more than about 2 times greater than the weight percent of the polyprenol pyrophosphates.

In still yet another embodiment, the present invention contemplates compound(s) of the present invention used in a method of modulating the functionality of integrins in a subject, wherein the weight percent of polyprenol monophosphates is not more than about 4 times greater than the weight percent of the polyprenol pyrophosphates.

In still yet another embodiment, the present invention contemplates compound(s) of the present invention used in a method of modulating the functionality of integrins in a subject, wherein the weight percent of polyprenol monophosphates is not more than about 5 times greater than the weight percent of the polyprenol pyrophosphates.

In still yet another embodiment, the present invention contemplates compound(s) of the present invention used in a method of modulating the functionality of integrins in a subject, wherein the weight percent of polyprenol monophosphates is not more than about 10 times greater than the weight percent of the polyprenol pyrophosphates.

In still yet another embodiment, the present invention contemplates compound(s) of the present invention used in a method of modulating the functionality of integrins in a subject, wherein the weight percent of polyprenol monophosphates is not more than about 20 times greater than the weight percent of the polyprenol pyrophosphates.

In still yet another embodiment, the present invention contemplates compound(s) of the present invention used in a method of modulating the functionality of integrins in a subject, wherein n is 11 in at least 80% of the polyprenol monophosphates present.

In still yet another embodiment, the present invention contemplates compound(s) of the present invention used in a method of modulating the functionality of integrins in a subject, wherein m is 11 in at least 80% of the polyprenol pyrophosphates present.

In still yet another embodiment, the present invention contemplates compound(s) of the present invention used in a method of modulating the functionality of integrins in a subject, wherein the weight percent of polyprenol monophosphates is not less than about 10 times greater than the weight percent of the polyprenol pyrophosphates.

In still yet another embodiment, the present invention contemplates compound(s) of the present invention used in a method of modulating the functionality of integrins in a subject, wherein the weight percent of polyprenol monophosphates is not less than about 20 times greater than the weight percent of the polyprenol pyrophosphates.

In still yet another embodiment, the present invention contemplates compound(s) of the present invention used in a method of modulating the functionality of integrins in a subject, wherein the integrins comprise CD11b and any other chain.

In still yet another embodiment, the present invention contemplates compound(s) of the present invention used in a method of modulating the functionality of integrins in a subject, wherein the activation of integrins is identified by the increase in the expression of CD11b.

In still yet another embodiment, the present invention contemplates compound(s) of the present invention used in a method of modulating the functionality of integrins to attenuate the proinflammatory cytokine burst.

In still yet another embodiment, the present invention contemplates compound(s) of the present invention used in a method of modulating the functionality of integrins in a subject, wherein R1 and or R2 and or R3 and or R4 are different moieties.

In still yet another embodiment, the present invention contemplates compound(s) of the present invention used in a method of modulating the functionality of integrins in a subject, wherein at least two of R1 and or R2 and or R3 and or R4 are the same moieties.

In still yet another embodiment, the present invention contemplates compound(s) of the present invention used in a method of modulating the functionality of integrins in a subject, wherein one or both R1 and R2 are not —CH3.

In regards to the teachings of this specification, in one embodiment, the present invention contemplates a method of modulating the response of innate immune system in a subject, said method comprising administering to or causing to be administered to the subject a pharmaceutical composition comprising at least one polyprenol phosphate selected from the general formulas:

R1—CH2(R2—CH2)—C═CH—CH2—[CH2—C(CH3)═CH—CH2]n-P(═O)(O)2—R3(R4)

R1(R2)—C═CH—CH2—[CH2—C(CH3)═CH—CH2]m-O—P(═O)(OH)—O—P(═O)(O)2—R3(R4)

wherein R1 and or R2 and or R3 and or R4 are selected from a group consisting of, —H, —OH, —CH3, hydrocarbons, amino acids, amines, lipids, carbohydrates or sugars and wherein in or n is an integer from 5-18 or a salt thereof.

In another embodiment, the present invention contemplates that the pharmaceutical composition used in a method, wherein pharmaceutical composition additionally comprises a pharmaceutically acceptable carrier.

In yet another embodiment, the present invention contemplates compound(s) of the present invention used in a method, wherein n is 8, 9, 10, 11, 12, or 13 in greater than 50% of the polyprenol monophosphates.

In still yet another embodiment, the present invention contemplates compound(s) of the present invention used in a method, wherein in is 8, 9, 10, 11, 12, or 13 in greater than 50% of the polyprenol pyrophosphates.

In still yet another embodiment, the present invention contemplates compound(s) of the present invention used in a method, wherein the weight percent of polyprenol monophosphates is greater than the weight percent of the polyprenol pyrophosphates.

In still yet another embodiment, the present invention contemplates compound(s) of the present invention used in a method, wherein the weight percent of polyprenol monophosphates is not more than about two times greater than the weight percent of the polyprenol pyrophosphates.

In still yet another embodiment, the present invention contemplates compound(s) of the present invention used in a method, wherein the weight percent of polyprenol monophosphates is not more than about four times greater than the weight percent of the polyprenol pyrophosphates.

In still yet another embodiment, the present invention contemplates compound(s) of the present invention used in a method, wherein the weight percent of polyprenol monophosphates is not more than about five times greater than the weight percent of the polyprenol pyrophosphates.

In still yet another embodiment, the present invention contemplates compound(s) of the present invention used in a method, wherein the weight percent of polyprenol monophosphates is not more than about 10 times greater than the weight percent of the polyprenol pyrophosphates.

In still yet another embodiment, the present invention contemplates compound(s) of the present invention used in a method, wherein the weight percent of polyprenol monophosphates is not more than about 20 times greater than the weight percent of the polyprenol pyrophosphates.

In still yet another embodiment, the present invention contemplates compound(s) of the present invention used in a method, wherein n is 11 in at least 80% of the polyprenol monophosphates present.

In still yet another embodiment, the present invention contemplates compound(s) of the present invention used in a method, wherein in is 11 in at least 80% of the polyprenol pyrophosphates present.

In still yet another embodiment, the present invention contemplates compound(s) of the present invention used in a method, wherein the weight percent of polyprenol monophosphates is not less than about 10 times greater than the weight percent of the polyprenol pyrophosphates.

In still yet another embodiment, the present invention contemplates compound(s) of the present invention used in a method, wherein the weight percent of polyprenol monophosphates is not less than about 20 times greater than the weight percent of the polyprenol pyrophosphates.

In still yet another embodiment, the present invention contemplates compound(s) of the present invention used in a method, wherein said attenuation of inflammation of the innate immune system includes the activation of integrins.

In still yet another embodiment, the present invention contemplates compound(s) of the present invention used in a method, wherein said integrin heterodimers comprise chain CD11b and any other integrin chain.

In still yet another embodiment, the present invention contemplates compound(s) of the present invention used in a method, wherein activation of integrins is identified by the increase in the expression of CD11b.

In still yet another embodiment, the present invention contemplates compound(s) of the present invention used in a method, wherein the integrin further comprises CD11b and any other integrin molecule.

In still yet another embodiment, the present invention contemplates compound(s) of the present invention used in a method, wherein R1 and or R2 and or R3 and or R4 are different moieties.

In still yet another embodiment, the present invention contemplates compound(s) of the present invention used in a method, wherein at least two of R1 and or R2 and or R3 and or R4 are the same moieties.

In still yet another embodiment, the present invention contemplates compound(s) of the present invention used in a method, wherein one or both R1 and R2 are not —CH3.

### BRIEF DESCRIPTION OF THE DRAWINGS

FIG. 1A: The expression of CD11b on monocytes of specific pathogen-free cats inoculated with feline herpesvirus strain SGE. The expression was measured by flow cytometry as mean fluorescence intensity (MFI) of the anti-CD11b antibodies in the blood of the infected cats treated with the substance (Experimental group, n=10). FIG. 1B: The expression of CD11b on monocytes of the infected cats treated with placebo (Control group, n=10). For each group, the measurements taken on days 0, 3, 7, and 14 were compared using Anova and T-tests. A statistically significant difference was found in the integrin expression in the experimental but not the placebo group (Table 1). Whisker margins indicate the minimum and maximum values, and crossbars indicate median values for the group. An open circle designates a suspected outlier.

Normalization of blood markers in four cats diagnosed with feline infectious peritonitis (FIP) and treated with the substance is illustrated in FIGS. 2A and 2B. All four cats were diagnosed by their veterinarians with non-effusive FIP based on clinical signs and laboratory findings. In all four cats, veterinarians obtained histologic, cytologic, or immunohistochemical evidence of pyogranulomatous inflammation. All cats had borderline to severe neutrophilia and abnormally low albumin/globulin (A/G) ratio along with other pathological changes in the bloodwork. All cats returned to being well and survived over 2 years after the diagnosis and the start of the treatment. In all cats, the bloodwork values normalized within two months of the treatment with the substance. FIG. 2A: Inflammation-associated neutrophilia resolved in all cats after 1-2 months of the treatment [normal range (dotted line)−1600-15000 cells/μl]. FIG. 2B: The albumin/globulin (A/G) ratio increased to normal [normal value (dotted line)≥0.8] in all cats within 2-3 months on the treatment.

Cytological examination of fine-needle aspirates (FNA) of mesenteric lymph nodes in Cat #103 revealed the resolution of FIP-related inflammation. FIG. 3A: Mesenteric lymph nodes aspirated three days before the start of the treatment showed a severe FIP-related inflammatory response. FIG. 3B: After 143 days of the treatment with the substance, a repeat aspiration of the same lymph nodes had no signs of inflammation.

### DETAILED DESCRIPTION OF THE INVENTION

### Formulations and Uses of the Compounds of the Present Invention

The inventors have discovered that compositions described in U.S. Pat. 9,872,867 to Kuril: (which is incorporated herein by reference) and variations (including both mono- and pyrophosphorylated variations) described herein have utility for the modulation of the innate immune response in general and the activation and modulation of the functionality of integrins in particular. In this regard, the compounds of the present invention may be used for the treatment and prevention of a wide range of infectious diseases caused by fungi, bacteria, viruses or used in oncologic pathology, for modulation of the innate immune response component of allergic or inflammatory diseases and for enhancement of the action of vaccines when used wholly or partly as an adjuvant, etc.

Specific and preferred values listed below are for illustration only; they do not exclude other defined values or other values within defined ranges for the radicals and substituents.

The polyprenol phosphates and pyrophosphates can be prepared from polyprenol using procedures similar to those known in the art or isolated from natural sources incorporated herein by reference to U.S. Pat. No. 9,872,867 to Kuritz.

Administration of the compounds as salts may be appropriate. Acceptable salts include alkali metal (for example, sodium, potassium, or lithium) or alkaline earth metal (for example, calcium) salts. However, any salt that is non-toxic and effective when administered to the animal or other organism being treated is acceptable.

Acceptable salts may be obtained using standard procedures well known in the art, for example, by reacting a sufficiently acidic compound with a suitable base affording a physiologically acceptable anion.

The compositions of the invention can be formulated as pharmaceutical compositions and administered to an animal host (or other organism) such as a human patient in a variety of forms adapted to the chosen route of administration, i.e., orally or parenterally (not via the digestive canal), by intravenous (i.v.), intramuscular (i.m.), topical, or subcutaneous routes, for example.

Thus, the present compound(s) may be systemically administered, e.g., orally, in combination with a pharmaceutically acceptable vehicle such as an inert diluent or an assimilable edible carrier. They may be enclosed in hard or soft shell gelatin capsules, may be compressed into tablets or may be incorporated directly with the food of the patient's diet. For oral therapeutic administration, the active compound(s) may be combined with one or more excipients and used in the form of ingestible tablets, buccal tablets, troches, capsules, elixirs, suspensions, syrups, wafers, and the like. Such compositions and preparations should contain at least 0.1% of the active compound by weight percent. The percentage of the compositions and preparations may, of course, be varied and may conveniently be between about 2 to about 60% of the weight of a given unit dosage form. The amount of active compound(s) in such therapeutically useful compositions is such that an effective dosage level will be obtained. When administered orally, the compositions of the invention can preferably be administered in a gelatin capsule.

The tablets, troches, pills, capsules, and the like may also contain the following: binders such as gum tragacanth, acacia, corn starch, or gelatin; excipients such as dicalcium phosphate; a disintegrating agent such as corn starch, potato starch, alginic acid, and the like; a lubricant such as magnesium stearate; and a sweetening agent such as sucrose, fructose, lactose or aspartame or a flavoring agent such as peppermint, oil of wintergreen or cherry flavoring may be added. When the unit dosage form is a capsule, it may contain, in addition to materials of the above type, a liquid carrier, such as vegetable oil or a polyethylene glycol. Various other materials may be present as coatings or to otherwise modify the physical form of the solid unit dosage form. For instance, tablets, pills, or capsules may be coated with gelatin, wax, shellac, sugar, and the like. A syrup or elixir may contain the active compound(s), sucrose or fructose as a sweetening agent, methyl and propylparabens as preservatives, a dye and flavoring, for example, cherry, tuna, or orange flavor and the like. Of course, any material used in preparing any unit dosage form should be pharmaceutically acceptable and substantially non-toxic in the amounts employed. In addition, the active compound(s) may be incorporated into sustained-release preparations and devices.

The compositions of the invention may also be administered intravenously or intraperitoneally by infusion or injection. Solutions of the active composition can be prepared in water, optionally mixed with a non-toxic surfactant and or alcohol. Dispersions can also be prepared in glycerol, liquid polyethylene glycols, triacetin, and mixtures thereof and in oils. Under ordinary storage and use conditions, these preparations may contain a preservative to prevent the growth of microorganisms.

The pharmaceutical dosage forms suitable for injection or infusion can include sterile aqueous solutions or dispersions or sterile dry material comprising the active ingredient which are adapted for the extemporaneous preparation of sterile injectable or infusible solutions or dispersions, optionally encapsulated in liposomes. In all cases, the ultimate dosage form should be sterile, fluid, and stable under manufacture and storage conditions. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers, or sodium chloride. Prolonged absorption of the injectable compositions can be brought about by the use in the compositions of agents delaying absorption, for example, aluminum monostearate and gelatin.

Sterile injectable solutions are prepared by incorporating the active composition in the required amount in the appropriate solvent with various other ingredients enumerated above, as needed, followed by filter sterilization. In the case of sterile powders for the preparation of sterile injectable solutions, the preferred methods of preparation are vacuum drying and freeze-drying techniques, which yield a powder of the active ingredient plus any additional desired ingredient present in the previously sterile-filtered solutions.

For topical administration, the present compositions may be applied in pure form, i.e., liquids. However, it will generally be desirable to administer them to the skin as compositions or formulations combined with a dermatologically acceptable carrier, which may be a solid or a liquid.

Useful solid carriers include finely divided solids such as talc, clay, microcrystalline cellulose, silica, alumina, and the like. Useful liquid carriers include water, alcohols, or glycols, or water-alcohol/glycol blends, in which the present compounds can be dissolved or dispersed at effective levels, optionally with the aid of non-toxic surfactants. Additives such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners, such as synthetic polymers, fatty acids, fatty acid salts and esters, fatty alcohols, modified celluloses or modified mineral materials can also be employed with liquid carriers to form spreadable pastes, gels, ointments, soaps and the like for application directly to the skin of the user.

Examples of useful dermatological compositions which can be used to deliver the compounds and derivatives of Formula 1 and or Formula 2 to the skin are known to the art; for example, see Jacquet, et al., (U.S. Pat. No. 4,608,392), Geria (U.S. Pat. No. 4,992,478), Smith el al., (U.S. Pat. No. 4,559,157) and Wortzman (U.S. Pat. No. 4,820,508), all of which are incorporated herein by reference.

Useful dosages of the compounds of Formula 1 and or Formula 2, or derivatives derived therefrom and described herein, can be determined by comparing their in vitro activity and in vivo activity in animal models. Methods for extrapolating effective dosages in mice and other animals to humans are known to the art; for example, see U.S. Pat. No. 4,938,949, which is incorporated herein by reference.

Generally, the concentration of the compositions of the invention in a liquid composition, such as a lotion, will be from about 0.1-50 wt % (weight percent), preferably from about 0.5-5 wt %. The concentration in a semi-solid or solid composition such as a gel or a powder will be about 0.1-5 wt %, preferably about 0.5-2.5 wt %.

The amount of the composition required for use in treatment will vary not only with the particular salt selected but also with the route of administration, the nature of the condition being treated, and the age and condition of the patient and will be ultimately at the discretion of the attendant physician or clinician.

In general, however, a suitable average dose will be in the range of from about 0.25 to about 100 mg/kg, e.g., from about 10 to about 75 mg/kg of body weight per day, such as 3 to about 50 mg per kilogram bodyweight of the recipient per day, preferably in the range of 6 to 90 mg/kg/day, most preferably in the range of 15 to 60 mg/kg/day.

The compositions are conveniently administered in a unit dosage form; for example, containing 5 to 1000 mg, conveniently 10 to 750 mg, most conveniently, 50 to 500 mg of active ingredient per unit dosage form.

Ideally, the active ingredient should be administered to achieve peak plasma concentrations of the active compound from about 0.5 to about 75 μM, preferably, about 1 to 50 μM, most preferably, about 2 to about 30 μM. This may be achieved, for example, by the intravenous injection of a 0.05 to 5 wt % solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, such as two, three, four, or more sub-doses per day. The sub-dose itself may be further divided, e.g., into several discrete loosely spaced administrations, such as multiple inhalations from an insufflator or by application of a plurality of drops into the eye.

Although the present invention is not limited to any specific theory, it is believed that the compositions of the invention are poly-functional both at the cellular and at the organism levels. At the cellular level, they are incorporated into or associated with the cellular membranes, enhancing their permeability and changing membrane properties, including the adhesion molecules. They also normalize and activate processes of cell surface glycoprotein biosynthesis, normalizing cell reproduction intracellular and, as a result, inter-tissue interactions. In the organism, on the whole, they normalize, augment or activate immune system functioning or modulate it through mechanisms associated with integrin signaling and, for example, integrin signaling to improve the function of individual organs, reduce pathological inflammation, reduce effects of stresses, enhance blood generation function and facilitate tissue regeneration.

The compositions of the invention are useful for prevention, treatment, and liquidation of consequences of diseases involving an innate immune response, including, but not limited to, viral, chlamydial, bacterial, oncology, liver, gastrointestinal, urologic and reproductive system, immune system, wounds, burns, and stresses.

The immunity modulating and anti-inflammatory activity of the compositions of the invention can be determined using assays that are known in the art or can be determined using assays similar to those described in the following examples.

The compositions of the invention can be used to treat numerous human viral diseases caused by, for example, coronaviruses, such as SARS-family, including SARS-CoV-2; and herpes viruses, such as but not limited to HSV-1, HSV-2, Herpes zoster, etc. Further, the compositions of the invention may be used to treat animal diseases caused by numerous viruses, including distemper virus (DV), canine enteritis (parvo, rota, and corona viruses; CEV), canine infectious hepatitis (CIH), feline infectious gastroenteritis (panleukopenia, FIE), feline infectious rhinotracheitis (agent—herpes virus; FHV), feline infectious enteritis and peritonitis (agent—coronavirus, FIP), COVID (SARS-2), feline leukemia (FeLV), feline panleukopenia (agent—parvovirua), feline immunodeficiency virus (FIV) , swine transmissive gastroenteritis (agent—rotavirus; STG), murine ectromelia (ME), cattle leukemia (CL), calf mixed viral infection (agents—parvo, adeno and coronaviruses; CMVI), western equestrian encephalomyelitis (WEE), and rabies (RV).

As used in the examples herein, the phrase “compounds of the present invention (Formula 1 and or Formula 2 and derivatives thereof)” is a composition of the invention wherein the compound(s) of the invention comprise at least 50 wt %, 75 wt %, 90 wt %, 95 wt %, 99 wt %, 99.5 wt % or 99.9 wt % of one or more of the compounds or derivatives described herein not including any additives, excipients or other ingredients that are not. In addition, the phrase “compounds of the present invention (Formula 1 and or Formula 2 and derivatives thereof)” also encompass any of the phosphorylated polyprenol-based compounds and compositions given herein as well as their stereoisomeric forms.

In this regard, the present invention contemplates compositions comprising,

1) polyprenol monophosphates of Formula 1:

R1—CH2(R2—CH2)—C═CH—CH2—[CH2—C(CH3)═CH—CH2]n-P(═O)(O)2—R3(R4)

wherein R1 and or R2 and or R3 and or R4 are selected from a group consisting of, —H, —OH, —CH3, hydrocarbons, amino acids, amines, lipids, carbohydrates or sugars and wherein n is an integer from 5-18 inclusive or a salt thereof, and or
2) polyprenol pyrophosphates of the Formula 2:

R1(R2)—C═CH—CH2—[CH2—C(CH3)═CH—CH2]m-O—P(═O)(OH)—O—P(═O)(O)2—R3(R4)

wherein R1 and or R2 and or R3 and or R4 are selected from a group consisting of, —H, —OH, —CH3, hydrocarbons, amino acids, amines, lipids, carbohydrates or sugars and wherein m is an integer from 5-18 inclusive or a salt thereof. The invention also provides pharmaceutical compositions comprising the compounds of the invention as well as therapeutic methods for using the compositions.

It will be appreciated by those skilled in the art that polyisoprenes and derivatives thereof possess double bonds, which may exist in cis or trans configurations. It is to be understood that the present invention encompasses any stereoisomeric form of the polyisoprenes chain of the present invention as well as mixtures thereof, which possess the useful properties described herein.

Specific and preferred values listed below are for illustration only; they do not exclude other defined values or other values within defined ranges for the radicals and substituents.

A specific composition of the invention is a composition wherein n is at least 6, wherein the polyprenol monophosphate comprises at least 90%, 95%, 99% or 99.5% of the weight of the composition and the polyprenol pyrophosphate comprises less 10% of the weight.

A specific composition of the invention is a composition wherein n is 8, 9, 10, 11, 12 or 13 in greater than 50% of the polyprenol monophosphates.

A specific composition of the invention is a composition wherein m is 8, 9, 10, 11, 12, or 13 in greater than 50% of the polyprenol pyrophosphates.

A specific composition of the invention is a composition wherein the weight percent of polyprenol monophosphates is greater than the weight percent of the polyprenol pyrophosphates.

A specific composition of the invention is a composition wherein the weight percent of polyprenol monophosphates is not more than about 2 times greater than the weight percent of the polyprenol pyrophosphates.

A specific composition of the invention is a composition wherein the weight percent of polyprenol monophosphates is not more than about 4 times greater than the weight percent of the polyprenol pyrophosphates.

A specific composition of the invention is a composition wherein the weight percent of polyprenol monophosphates is not more than about 5 times greater than the weight percent of the polyprenol pyrophosphates.

A specific composition of the invention is a composition wherein the weight percent of polyprenol monophosphates is not more than about 10 times greater than the weight percent of the polyprenol pyrophosphates.

A specific composition of the invention is a composition wherein the weight percent of polyprenol monophosphates is not more than about 20 times greater than the weight percent of the polyprenol pyrophosphates.

A specific composition of the invention is a composition wherein n is 10 in at least 80% of the polyprenol monophosphates present.

A specific composition of the invention is a composition wherein m is 10 in at least 80% of the polyprenol pyrophosphates present.

A specific composition of the invention is a composition wherein n is 10 in at least 80% of the polyprenol monophosphates present, and m is 10 in at least 80% of the polyprenol pyrophosphates present.

A specific composition of the invention is a composition wherein n is 10 in at least 80% of the polyprenol monophosphates present, m is 10 in at least 80% of the polyprenol pyrophosphates present, and the weight percent of polyprenol monophosphates is about 10 times greater than the weight percent of the polyprenol pyrophosphates.

A specific composition of the invention is a composition wherein n is 10 in at least 90% of the polyprenol monophosphates present.

A specific composition of the invention is a composition wherein m is 10 in at least 90% of the polyprenol pyrophosphates present.

A specific composition of the invention is a composition wherein n is 10 in at least 90% of the polyprenol monophosphates present, and m is 11 in at least 90% of the polyprenol pyrophosphates present.

A specific composition of the invention is a composition wherein n is 10 in at least 90% of the polyprenol monophosphates present, m is 10 in at least 90% of the polyprenol pyrophosphates present, and the weight percent of polyprenol monophosphates is about 10 times greater than the weight percent of the polyprenol pyrophosphates.

The target and the mechanism of the effect that can define the utility of the compositions of the invention can be determined using assays that are known in the art or can be determined using assays similar to those described in the following examples.

The applicant has discovered certain compositions that are useful for the prevention and or treatment of diseases, including viral-, chlamydial-, bacterial-related diseases, oncology-related diseases, diseases related to the liver, gastrointestinal, urologic, and reproductive systems, and diseases related to the function of the immune system. In this regard, the present invention contemplates that the compositions of the present invention are also effective in the treatment of wounds, burns, allergic diseases, and stresses and are useful for medical (human), veterinary, and agricultural applications. The phosphorylated compositions of the invention have improved solubility compared to related known compositions and, as a result, demonstrate improved levels of activity against certain diseases.

In particular, it is contemplated that the compounds of the present invention are effective in the modulation (i.e., activation, attenuation, and other ways of regulation) of the innate immune system through, for example, interactions involving (directly or indirectly) integrins. Furthermore, it is contemplated that the compounds, through the direct or indirect interaction with integrins, attenuate pathologic inflammation caused by the damages to the immune system by whereas sustaining host-defense.

### Integrins, Innate Immunity, and Pathologic Inflammation

The invention also provides a method for modulating an innate immune response and or integrin-mediated immune response effects and or attenuating pathological inflammation in an animal comprising administering to an animal in need of such treatment, an effective amount of a composition of the comprising at least one compound of the present invention. As used herein, “animal” includes, for example, mammals (e.g., a dog, cow, cat, or human), birds (e.g., poultry), and other animals (fish, insects, etc.) that can effectively be treated with the compositions of the invention. In this regard, integrins are highly conserved molecules in the animal kingdom. In fact, the compounds of the present invention have been shown to be effective in bees.

As used in the examples herein, the phrase “compounds of the present invention (Formula 1 and or Formula 2 and derivatives thereof)” is a composition of the invention wherein the compound(s) of the invention comprise at least 50 wt %, 75 wt %, 90 wt %, 95 wt %, 99 wt %, 99.5 wt % or 99.9 wt % of one or more of the compounds or derivatives described herein not including any additives, excipients or other ingredients that are not. In addition, the phrase “compounds of the present invention (Formula 1 and or Formula 2 and derivatives thereof)” also encompass any of the phosphorylated polyprenol-based compounds and compositions provided herein as well as their stereoisomeric forms.

It will be appreciated by those skilled in the art that polyisoprenes possess double bonds, which may exist in cis or trans configurations. It is to be understood that the present invention encompasses any stereoisomeric form of the polyisoprenes of the present invention as well as mixtures thereof, which possess the useful properties described herein.

Specific and preferred values listed below are for illustration only; they do not exclude other defined values or other values within defined ranges for the radicals and substituents.

In this regard, the present invention contemplates compositions comprising,

1) polyprenol monophosphates of Formula 1:

R1—CH2(R2—CH2)—C═CH—CH2—[CH2—C(CH3)═CH—CH2]n-P(═O)(O)2—R3(R4)

wherein R1 and or R2 and or R3 and or R4 are selected from a group consisting of, —H, —OH, —CH3, hydrocarbons, amino acids, amines, lipids, carbohydrates or sugars and wherein n is an integer from 5-18 inclusive or a salt thereof, and or
2) polyprenol pyrophosphates of the Formula 2:

R1(R2)—C═CH—CH2—[CH2—C(CH3)═CH—CH2]m-O—P(═O)(OH)—O—P(═O)(O)2—R3(R4)

wherein R1 and or R2 and or R3 and or R4 are selected from a group consisting of, —H, —OH, —CH3, hydrocarbons, amino acids, amines, lipids, carbohydrates or sugars and wherein m is an integer from 5-18 inclusive or a salt thereof. The invention also provides pharmaceutical compositions comprising the compounds of the invention as well as therapeutic methods for using the compositions.

A specific composition of the invention is a composition wherein n is at least 6, wherein the polyprenol monophosphate comprises at least 90%, 95%, 99% or 99.5% of the weight of the composition, and the polyprenol pyrophosphate comprises less 10% of the weight.

A specific composition of the invention is a composition wherein n is 8, 9, 10, 11, 12 or 13 in greater than 50% of the polyprenol monophosphates.

A specific composition of the invention is a composition wherein m is 8, 9, 10, 11, 12, or 13 in greater than 50% of the polyprenol pyrophosphates.

A specific composition of the invention is a composition wherein the weight percent of polyprenol monophosphates is greater than the weight percent of the polyprenol pyrophosphates.

A specific composition of the invention is a composition wherein the weight percent of polyprenol monophosphates is not more than about 2 times greater than the weight percent of the polyprenol pyrophosphates.

A specific composition of the invention is a composition wherein the weight percent of polyprenol monophosphates is not more than about 4 times greater than the weight percent of the polyprenol pyrophosphates.

A specific composition of the invention is a composition wherein the weight percent of polyprenol monophosphates is not more than about 5 times greater than the weight percent of the polyprenol pyrophosphates.

A specific composition of the invention is a composition wherein the weight percent of polyprenol monophosphates is not more than about 10 times greater than the weight percent of the polyprenol pyrophosphates.

A specific composition of the invention is a composition wherein the weight percent of polyprenol monophosphates is not more than about 20 times greater than the weight percent of the polyprenol pyrophosphates.

A specific composition of the invention is a composition wherein n is 10 in at least 80% of the polyprenol monophosphates present.

A specific composition of the invention is a composition wherein m is 10 in at least 80% of the polyprenol pyrophosphates present.

A specific composition of the invention is a composition wherein n is 10 in at least 80% of the polyprenol monophosphates present, and m is 10 in at least 80% of the polyprenol pyrophosphates present.

A specific composition of the invention is a composition wherein n is 10 in at least 80% of the polyprenol monophosphates present, m is 10 in at least 80% of the polyprenol pyrophosphates present, and the weight percent of polyprenol monophosphates is about 10 times greater than the weight percent of the polyprenol pyrophosphates.

A specific composition of the invention is a composition wherein n is 10 in at least 90% of the polyprenol monophosphates present.

A specific composition of the invention is a composition wherein m is 10 in at least 90% of the polyprenol pyrophosphates present.

A specific composition of the invention is a composition wherein n is 10 in at least 90% of the polyprenol monophosphates present, and m is 11 in at least 90% of the polyprenol pyrophosphates present.

A specific composition of the invention is a composition wherein n is 10 in at least 90% of the polyprenol monophosphates present, m is 10 in at least 90% of the polyprenol pyrophosphates present, and the weight percent of polyprenol monophosphates is about 10 times greater than the weight percent of the polyprenol pyrophosphates.

The target and the mechanism of the effect that can define utility of the compositions of the invention can be determined using assays that are known in the art or can be determined using assays similar to those described by means of the following non-limiting examples.

### EXEMPLIFICATION

### Example 1: Upregulation of Integrin CD11b in Cats Inoculated With the Feline Herpesvirus and Treated With the Compositions But Not With Placebo

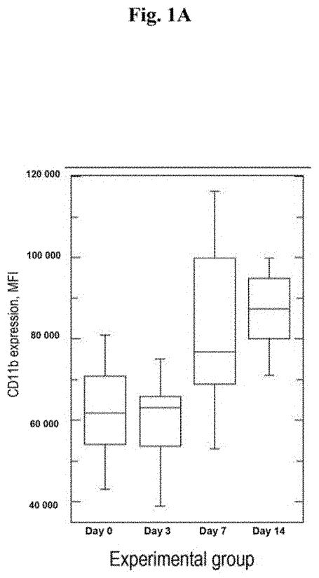

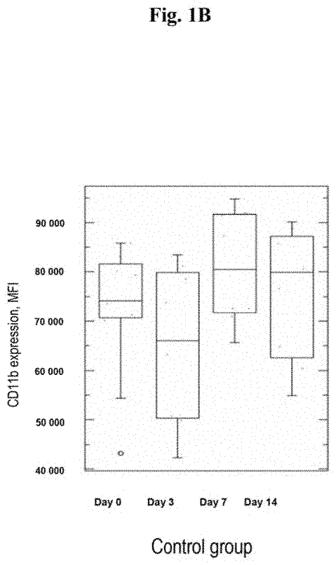

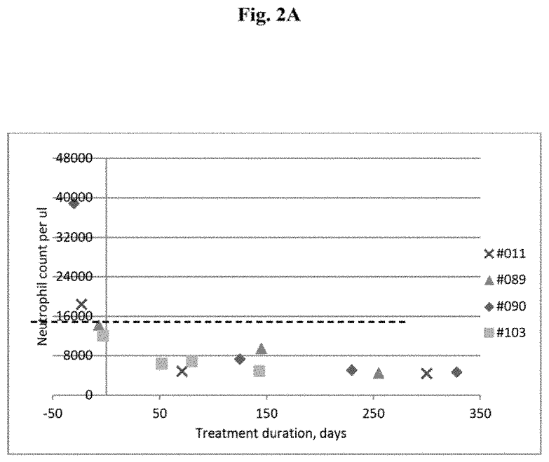

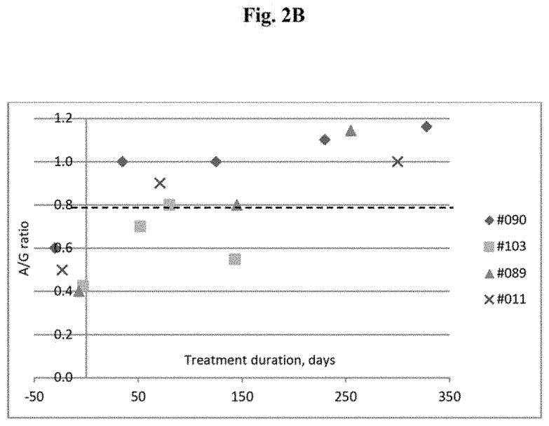

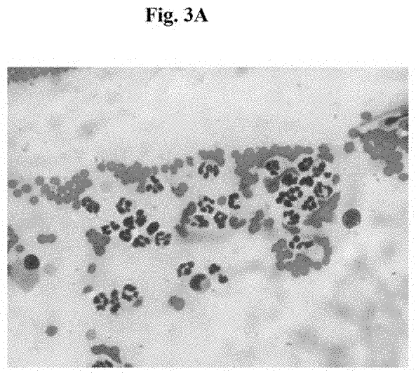

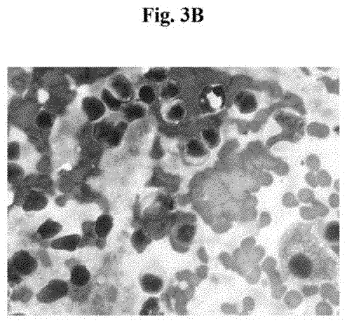

The Compounds of Formula 1 and Formula 2 and the derivatives thereof of the present invention effectively upregulated the expression of integrins CD11b on monocytes of cats inoculated with feline herpesvirus. The cats receiving a placebo did not show the upregulation of the integrins (FIG. 1, Table 1).

A representative phosphorylated compound of the present invention (as described in U.S. Pat. 9,872,867, which is herein incorporated by reference: “Substance”) was obtained from Sass & Sass, Inc. (Oak Ridge, Tenn.).

The double-blinded placebo-controlled trial in cats experimentally inoculated with feline herpesvirus is described by Legendre et al. (2017, Front. Vet. Sci., 4: 24), which is herein incorporated by reference. The specific pathogen-free cats were inoculated with feline herpesvirus strain SGE (US Department of Agriculture). A statistically significant difference was found in the integrin expression in the blood of the infected cats treated with the substance (Panel A. Experimental group, n=10) but not in the infected cats treated with placebo (Panel B. Control group, n=10). Whisker margins indicate the minimum and maximum values, and crossbars indicate median values for the group. An open circle designates a suspected outlier.

At the time of the study, blood was drawn from the jugular vein of kittens on days 0, 3, 7, and 14 post-inoculation and filled into sodium heparin-treated Vacutainer tubes. The tubes were gently inverted, and 3×100 μL aliquots from each sample were fixed with 4% paraformaldehyde for 10 minutes to preserve the surface markers. The samples were washed with PBS, topped off with 2% horse serum, and centrifuged for 10 min at 300g.

The integrin CD11b is constitutively expressed at low levels when they are not activated Martinez et al., J. Immunol., 2020, 205: 2545-2553, doi: 10.4049/jimmuno1.1901402); however, when the cells are activated, the expression of CD11b increases. The expression of CD11b integrin was measured on monocytes by flow cytometry as mean fluorescence intensity (MFI) of the anti-CD11b antibodies. The measurements received on days 0, 3, 7, and 14 were compared using Anova and T-tests (Table 1). We analyzed the expression of CD11b using blood samples collected on 0, 3, 7, and 14 DPC from the cats in both groups. The effect of the treatment was established by the changes in fluorescence of corresponding antibodies in flow cytometry. Pellets were resuspended in 100 μL of 1× PBS and incubated with 0.5 μg of each primary antibody specific for CD14 (R&D Systems) and CD11b (AbCam) for one h at room temperature. The pellets in tubes were washed with PBS, centrifuged, and resuspended in 100 μL of PBS as before. Fluorescent secondary antibodies (0.5 μg of each) specific for the CD14 in thus restricting the signal to monocytes (Alexa 488; Jackson Immunoresearch) and CD11b (Alexa 647; Jackson Immunoresearch) primaries were added to each tube and incubated for 45 min at room temperature. Corresponding isotypic controls were used as negative controls at the same concentration as the primaries. Cells were washed with PBS, centrifuged, and resuspended in 1 mL of red blood cell lysis solution (BD FACS Lyse, BD Biosciences,). After 10 min, cells were centrifuged as before and resuspended in 300 μL of PBS.

The samples were analyzed using an Accuri C6 flow cytometer (BD Biosciences), and 50,000 events were collected per sample. An initial gate was placed around the monocyte population from the whole blood profile of forwarding vs. side scatter. Secondary gates were applied to the isotypic control and set to 1% positive fluorescence intensity. The mean fluorescence intensity (MFI) for CD11b was obtained from those events within the same gates in all samples. MFI is proportional to the number of receptors per cell.

We measured the MFI inside the above-described gated in blood collected on days 0, 3, 7, and 14 from cats in the experimental and control groups and used Anova and T-tests for statistical analysis (Table 1). FIG. 1 shows intragroup changes in the mean fluorescence intensity (MFI) of CD11b-tagged monocytes in the blood of cats inoculated with FRV and administered either the substance or placebo. We observed individual variability in the baseline CD11b expression between cats as well as different times required for the CD11b upregulation. Within the group treated with the substance, the increase in the integrin levels was continuous and significant between the background (Day 0) to Days 7 and 14 (FIG. 1A, Table 1). Cats treated with placebo no upregulation of the integrin: no significant difference was observed between the integrin expression on day 0 and any other day (FIG. 1B, Table 1). Absolute MFI values became significantly different between the groups on Day 14 (p=0.002, T-test).

TABLE 1
Significance values (p) for tests of CD11b activation
in control and experimental groups

| | | t-Test | | |
|---|---|---|---|---|
| | | Day 0 vs. | Day 0 vs. | Day 0 vs. |
| Group | ANOVA (p) | Day 3 | Day 7 | Day 14 |
| Experimental | <0.0001* | 0.765 | 0.003* | <0.0001* |
| Placebo/control | 0.10** | 0.658** | 0.099 | 0.510 |

*Significant difference
**n = 9 (data for one cat were unavailable on Day 3)

All inoculated cats of both groups developed signs of the disease. However, cumulative clinical scores in the group treated with the substance were significantly lower than in cats treated with placebo; the separation of the scores between the groups became significant at Days 7-14 post-inoculation (Legendre et al., Front. Vet. Sci., 2017, 4: 24). The increase in the difference in CD11b expression between the groups paralleled the increase in the separation of clinical scores between the groups. The obvious parallelism in the timing of the separation of clinical scores and monocyte activation suggested the link between those events.

### Example 2: Changes in the Markers of Inflammation in the BBood of Cats With Feline Infectious Peritonitis, a Naturally Occurring Coronaviral Disease

Feline infectious peritonitis (FIP) is a fatal disease caused by a mutated feline enteric coronavirus (FCoV) which triggers global immunity dysregulation in a cat (Pedersen, 2014, The Vet. J. 201: 123-132). The pathological inflammation is a result of the overproduction of proinflammatory cytokines (ibid.) and perhaps integrins (Acar et al., J. Gen. Virol., 2016, 97: 2633-2642); it causes many pathologies, including pyogranulomatous inflammation on the internal organs; the latter is considered specific to the disease (Pedersen, ibid.).

FIG. 2. Normalization of blood markers in four cats diagnosed with feline infectious peritonitis (FIP) and treated with the substance.

Cat #011. A 11 y.o. Persian, neutered female, presented with anorexia, lethargy, fever not responsive to antibiotic therapy, mesenteric lymph node enlargement. Hematology revealed neutrophilia, anemia, and other abnormalities; chemistry showed albumin to globulin ratio (A/G)=0.5. The cat was negative for FeLV/FIV and other agents on the anemia panel. The biopsies of multiple organs were submitted for histopathological analysis, which showed inflammation: granulomatous lymphoadenitis, “lymphohistiocytic portal hepatitis, and phlebitis, noted with FIP.” The cat was diagnosed with non-effusive FIP and started treatment with the 3 mg/kg of substance three times weekly; the clinical signs resolved, weight increased, and within 60 days of the treatment, the blood values returned to normal and remained normal for the duration of the treatment. The cat was doing well for 2.5 years and was then lost to follow-up.

Cat #089. A 6 months-old Siberian, intact male, presented with fever not responsive to antibiotics, anterior synechia, uveitis. After ruling out Toxoplasma and Bartonella infections, the cat tested positive on FIP 7b ELISA test. The bloodwork showed inflammation: neutrophilia, eosinophilia, anemia, hyperglobulinemia, and other changes consistent with FIP. The substance was prescribed at 3 mg/kg 3 times weekly. The cat's condition improved, and blood values became normal. The cat was neutered and continued doing well over the next two years until his treatment was stopped. The cat survived 1166 days after the diagnosis.

Cat #090. A 6 months-old domestic shorthair, neutered male, presented for evaluation for inappetance and lethargy. Bloodwork revealed a leukocytosis characterized by a neutrophilia and monocytosis, and anemia, and a physical exam showed cervical lymphadenomegaly. The lymph node biopsy revealed inflammation (polygranulomatous lymphadenitis), and the tissue tested positive for FCoV mRNA. The combined histopathological and immunohistochemical results were most consistent with the diagnosis of non-effusive feline infectious peritonitis (FIP). The cat started treatment with the substance at 3 mg/kg every other day. The cat's condition improved, clinical signs resolved, and blood values became normal within 40 days; the masses were no longer palpable in the abdomen. After a year, the treatment was gradually decreased and discontinued. The cat is alive and keeps doing well at the time of this patent application, 6 years after the diagnosis.

Cat #103. A 23 month-old Oriental Shorthair, neutered male, neutered male, presented with a weight loss, inappetance, lethargy. Physical examination revealed enlarged abdominal lymph nodes, and the bloodwork showed hyperglobulinemia, borderline neutrophilia, and other changes. Fine needle aspirate of the lymph node showed inflammation was suggestive of the occult viral process, such as FIP. The cat started the treatment with the substance at 3 mg/kg 3 times weekly. After two months of the treatment, the cat returned to normal routines and doing well; the mild lymphadenopathy was palpated and appeared on the ultrasound, but it improved from the previous test (became smaller in size), and a larger mass was no longer present. After three months of treatment, no palpable masses were present in the abdomen. The treatment was discontinued after 1.5 years, and the cat is alive and has been doing well for 5 years since the diagnosis.

### Example 3. Resolution of Pyogranulomatous Inflammation on the Lymph Nodes in Cat #103 Cat Diagnosed With FIP and Treated With the Substance

In Cat #103 (see Example 2), fine needle aspiration (FNA) of mesenteric lymph nodes of Cat #103 has been performed three days before and after 143 days of the treatment with the substance. FIG. 3 shows the cytologic evidence of the resolution of FIP-related pyogranulomatous inflammation. A. Cytology of material collected three days before the start of the treatment shows “scattered throughout <. . . > moderate to high numbers of mixed inflammatory cells. <. . . > Diagnosis: neutrophilic pyogranulomatous inflammation”, the evidence of a severe inflammatory response before the treatment with the substance. B. Cytology of material collected by the FNA made after 143 days of the treatment with the substance shows no signs of pyogranulomatous inflammation, indicating that the treatment with the substance resolved the pathological inflammation.

We Claim:

1. A method of upregulating integrin CD11b on monocytes in a subject said method comprising administering, to or causing to be administered to the subject a pharmaceutical composition comprising at least one polyprenol phosphate selected from general formulas (1) or (2):
R1—CH2(R2—CH2)—C═CH—CH2—[CH2—C(CH3)═CH—CH2]n-P(═O)(O)2—R3(R4)
R1(R2)—C═CH—CH2—[CH2—C(CH3)═CH—CH2]m-O—P(═O)(OH)—O—P(═O)(O)2—R3(R4)
wherein R1 and or R2 and or R3 and or R4 are selected from a group consisting of, —H, —OH, —CH3, hydrocarbons, amino acids, amines, lipids, carbohydrates or sugars and wherein in or n is an integer from 5-18 or a salt thereof.

2. The method of claim 1, wherein the pharmaceutical composition additionally comprises a pharmaceutically acceptable carrier.

3. The method of claim 1, wherein a is 8, 9, 10, 11, 12, or 13 is greater than 50% of the polyprenol monophosphates.

4. The method of claim 1, wherein m is 8, 9, 10, 11, 12, or 13 in greater than 50% of the polyprenol pyrophosphates.

5. The method of claim 1, wherein the weight percent of polyprenol monophosphates is greater than the weight percent of the polyprenol pyrophosphates,

6. The method of claim 1, wherein the weight percent of polyprenol monophosphates is not more than about 2 times greater than the weight percent of the polyprenol pyrophosphates.

7. The method of claim 1, wherein the weight percent of polyprenol monophosphates is not more than about 4 times greater than the weight percent of the polyprenol pyrophosphates.

8. The method of claim 1, wherein the weight percent of polyprenol monophosphates is not more than about 5 times greater than the weight percent of the polyprenol pyrophosphates.

9. The method of claim 1, wherein the weight percent of polyprenol monophosphates is not more than about 10 times greater than the weight percent of the polyprenol pyrophosphates.

10. The method of claim 1, wherein the weight percent of polyprenol monophosphates is not more than about 20 times greater than the weight percent of the polyprenol pyrophosphates.

11. The method of claim 1, wherein n is 11 in at least 80% of the polyprenol monophosphates present.

12. The method of claim 1, wherein m is 11 in at least 80% of the polyprenol pyrophosphates present.

13. The method of claim 1, wherein the weight percent of polyprenol monophosphates is not less than about 10 times greater than the weight percent of the polyprenol pyrophosphates.

14. The method of claim 1, wherein the weight percent of polyprenol monophosphates is not less than about 20 times greater than the weight percent of the polyprenol pyrophosphates.

15. The method of claim 1, wherein said integrins comprise CD11b and any other integrin chain.

16. The method of claim 1, wherein modulation of the immune response is identified by the increase in the expression of integrin chain CD11b.

17. The method of claim 1, wherein R1 and or R2 and or R3 and or R4 are different moieties.

18. The method of claim 1, wherein at least two of R1 and or R2 and or R3 and or R4 are the same moieties.

19. The method of claim 1, wherein one or both R1 and R2 are not —CH3.

20. The method of claim 1, wherein said integrins comprise heterodimers of CD11b and any other chain.

21 . The method of claim 1, where said upregulation comprises signal transduction between immune cells.

22. The method of attenuating pathological inflammation in a subject, said method comprising administering to or causing to be administered to the subject a pharmaceutical composition comprising at least one polyprenol phosphate selected from the general formulas:
R1—CH2(R2—CH2)—C═CH—CH2—[CH2—C(CH3)═CH—CH2]n-P(═O)(O)2—R3(R4)
R1(R2)—C═CH—CH2—[CH2—C(CH3)═CH—CH2]m-O—P(═O)(OH)—O—P(═O)(O)2—R3(R4)
wherein R1 and or R2 and or R3 and or R4 are selected from a group consisting of, , OH, —CH3, hydrocarbons, amino acids, amines, lipids, carbohydrates or sugars and wherein m or n is an integer from 5-18 or a salt thereof or their modifications listed in claims 2-18.

23. The method of claim 22, wherein said pathological inflammation is due to the burst of proinflammatory cytokines, chemokines, adhesion molecules, and other elements of the innate immunity.

24. The method of claim 22, wherein said pathological inflammation results in the formation of neoplastic structures, such as but not limited to pyogranulomas, and other neoplasies, and said attenuation includes the resolution of those neoplastic elements.

26. The method of claim 22, wherein said pathological inflammation causes pathological changes in bloodwork but not limited to markers of hematopoiesis and liver function.

26. The method of claim 22 wherein pathological inflammation is caused to the dysregulation of the immune response due to stress, infection, or any other adverse condition.

27. The method of claim 26 wherein attenuation of the pathological inflammatory response is identified by the normalization of hematopoietic and other physiological functions.

28. The method of claim 26, wherein the normalization of hematopoietic and other physiological functions is identified by normalization and resolution of but not limited to neutrophilia, anemia, leukopenia, leukocytosis, monocytosis, etc., decrease in albumins, increase in globulins, pathological changes in liver enzymes, etc.

29. The method of claim 26, wherein the normalization of biochemical processes and function of corresponding organs is identified by normalization and resolution of but not limited to pathological levels of albumins, globulins, liver enzymes. etc.
